(19) United States
(12) Patent Application Publication
(10) Pub. No.: US 2003/0133990 A1
(43) Pub. Date: Jul. 17, 2003

(54) Bandage using molecular sieves

(76) Inventors: Francis X. Hursey, West Hartford, CT (US); Alan Wu, West Simsbury, CT (US); Steven L. Suib, Storrs, CT (US); Sandra L. Bushmich, Storrs, CT (US); Jia Liu, Storrs, CT (US); Beatriz Hincapie, Storrs, CT (US)

Correspondence Address: Alix, Yale & Ristas, LLP, Suite 1400, 750 Main Street, Hartford, CT, 06103-2721, US

(21) Appl. No.: 10/280,145

(22) Filed: Oct. 25, 2002

Related U.S. Application Data
(63) Continuation of application No. 09/687,127, filed on Oct. 13, 2000, now abandoned.

(51) Int. Cl. A61K 33/42; A61K 33/24; A61K 33/26; A61K 33/06

(52) U.S. Cl. 424/601; 424/653; 424/646; 424/617; 424/682

(57) ABSTRACT

A material for the enhancement of blood coagulation. The material comprises calcium cations, an inorganic oxide, a clay, an inorganic oxide in combination with calcium cations, a zeolite in combination with calcium cations, a zeolite in combination with an inorganic oxide and combinations thereof. The material when combined with blood reduces the coagulation time of the blood. Also, a method for using the material to promote blood coagulation.

### BACKGROUND OF THE INVENTION

This invention relates generally to wound dressings or coverings. More particularly, the present invention relates to the use of molecular sieve materials in wound dressings or coverings for the control of bleeding.

The occurrence of an accident involving the creation of a wound to the skin and circulatory system is unfortunately well known. Such wounds are often accompanied by bleeding. In minor accidents, the bleeding may be controlled by the body's own blood clotting mechanisms. In more severe wounds, the bleeding may be additionally controlled by the use of wound elevation, applied pressure and absorbent dressings or pads. These methods, either singly or in combination, may be ineffective for the control of bleeding from severe wounds or from persons with lessened blood clotting mechanisms.

Limited materials are known for the control of bleeding. One organic material is a microfibrillar collagen hemostat. While this material is effective for control of bleeding, it is very expensive. Alternatively, U.S. Pat. No. 4,822,349 teaches a method for reducing the flow of blood by applying a sterilized, dehydrated zeolite material to an opening from which the blood is emanating. It should be understood that the above materials, by themselves, are not part of the present invention.

### SUMMARY OF THE INVENTION

Briefly stated, the invention in a preferred form is a blood coagulation accelerator that is used to promote the rate of blood clotting. The blood coagulation accelerator may be directly applied to a wound or used as a film, coating or filler in the preparation of a wound cover or dressing. The use of the terms wound cover or dressing is not meant to be limiting and would include, for example, single layer covers such as gauze, multiple layer covers, multiple layer gauze pads which may include impermeable protective layers or covers or an envelope or sock formed of a blood permeable fabric within which the blood coagulation accelerator is retained. Application of the blood coagulation accelerator materials, either discreetly or as a film or coating in a wound dressing, speeds up the rate of blood clotting to arrest bleeding from the wound.

The blood coagulation accelerator preferably comprises a clay material, a molecular sieve material, an inorganic oxide material or combinations thereof. Without wishing to be bound to any theory, it is thought that at least the molecular sieve materials selectively absorb small molecules such as water in blood. The absorption of small molecules increases the rate of blood clotting. Further, the dehydration reactions generally evolve heat, which is helpful in arresting wound bleeding. Other applications for the inventive blood coagulation accelerator materials include self-cauterization and improved wound healing.

Preferably, the molecular sieve and inorganic oxide materials incorporate Ca ions. The incorporation of Ca ions into molecular sieve and inorganic oxide materials is shown to increase the effectiveness of such materials in arresting wound bleeding.

The molecular sieve and inorganic materials may be mixed and used in combination to markedly decrease the time of bleeding. The mixed materials are cheaper to produce and more effective in stopping bleeding than other currently available materials.

The inventive blood coagulation accelerator materials may be used in veterinary applications such as, for example, nail bleeding in dogs, cat declawing and veterinary surgery. The inventive blood coagulation accelerator materials may be used in human applications such as, for example, to stop epistaxis and hemorrhage related to low platelet numbers, hemophilia, during removal of intravenous catheters and to treat wounds incurred during accidents or military operations. It should be noted that the inventive blood coagulation accelerator materials are as effective as commercially available hemostat materials but can be less expensive to produce.

An object of the invention is to provide a material that will promote blood clotting.

Another object of the invention is to provide a material that can inexpensively speed the rate of blood clotting.

A further object of the invention is to provide a material that may be incorporated into a covering or dressing used to control bleeding.

A better understanding of the invention will be obtained from the following detailed disclosure of the article and the desired features, properties, characteristics, and the relation of the elements as well as the process steps, one with respect to each of the others, as set forth and exemplified in the description and illustrative embodiments.

### DETAILED DESCRIPTION

The disclosure of U. S. Pat. No. 4,822,349, issued Apr. 18, 1989, is incorporated by reference herein.

A number of blood coagulation accelerator materials were mixed with fresh blood samples using the blood of different animals from several animal species, including horse, cow and dog. The materials and blood were mixed in predetermined ratios and the time at which the blood in the test mixture clotted was recorded. This type of clotting test is called “the whole-blood coagulation test” and is widely performed, such as in monitoring heparin therapy. This type of test is neither the most sensitive or most precise clotting test known. However, in the present application this test allowed rapid and inexpensive screening of materials that exhibited increased blood coagulation effects from those materials that exhibited little or no blood coagulation effects. While animals were used as test subjects it is believed human blood will react in substantially the same fashion. Therefore the invention is applicable to both veterinary and human use.

Without wishing to be bound to any theory, it is thought that some blood coagulation accelerator materials selectively absorb small molecules such as water in blood. The absorption of small molecules increases the rate of blood clotting. Further, the dehydration reactions generally evolve heat, which is helpful in arresting wound bleeding. Preferably, the molecular sieve and inorganic oxide materials incorporate Ca ions. The incorporation of Ca ions into molecular sieve and inorganic oxide materials is shown to increase the effectiveness of such materials in arresting wound bleeding.

It should be noted that the results of this test procedure vary somewhat with different observers for the same blood coagulation accelerator materials. Additionally, some blood coagulation accelerator materials such as MgO form a slurry when mixed with blood, making determination of the clotting time difficult.

Some of the blood coagulation accelerator materials that lowered blood-clotting time were mixed, and these mixtures were tested for effect on blood clotting. Additionally, some blood coagulation accelerator materials were coated on various substrates or contained within packages. Blood coagulation accelerator materials were also used to control bleeding in animals. The blood coagulation accelerator materials were either obtained commercially or prepared in the laboratory. Materials commercially available are AVITENE, a microfibrillar collagen hemostat available from Davol Inc. of Cranston R.I.; (Na) zeolite 4A, ((Na)₁₂[(AlO₂)₁₂(SiO₂)₁₂].27H₂O), available under the trade name PURMOL 4A from Zeochem of Louisville, K.Y.; (Ca) zeolite 5A ((Ca)₆[(AlO₂)₁₂(SiO₂)₁₂].27H₂O), available under the trade name LINDE TYPE A from Union Carbide; Yunnan White Medicine available from China; MAXWELL HOUSE coffee available from Kraft; sodium aluminum oxide (NaAlO₂), available from Alfa Aesar Company of Ward Hill, Mass.; magnesium oxide (MgO), calcium oxide (CaO), phosphorus pentoxide (P₂O₅), acid silica (SiO₂.nH₂O), Chromatographic silica gel (SiO₂), calcium chloride (CaCl₂), secondary dibasic calcium phosphate (CaHPO₄), titanium dioxide (TiO₂), available as TITANIC OXIDE, and barium oxide (BaO), all available from Fisher Scientific Company; activated carbon, available from Strem Chemicals of Newburyport Mass.; europium oxide (Eu₂O₃) and cerium oxide (CeO₂), available from American Potash & Chemical Corp. of West Chicago, Ill.; copper oxide (CuO), available from Cerac Inc. of Milwaukee, Wis.; cobalt oxide (Co₂O₃), available from J. T. Baker; bismuth oxide (Bi₂O₃), available from Baker and Adamson Chemical Co.; aluminum oxide (Al₂O₃), available as aluminum oxide neutral type T from EM Reagents; nickel oxide (NiO), available from Matheson, Coleman and Bell of East Rutherford, N.J.; zinc oxide (ZnO), stannic oxide (SnO₂), and iron oxide, (Fe₂O₃) all available from Baker Analyticals; manganese oxide (MnO), available as manganese IV oxide, 99% and zirconium (IV) oxide (ZrO₂), all available from Aldrich; vanadium pentoxide (V₂O₅), available from Mallinkrodt; scandium oxide (Sc₂O₃), available as scandium oxide 98% from A. D. Mackay of New York; yttrium oxide (Y₂O₃), available from Alfa Aesar Company of Ward Hill, Mass.; CHROMOSORB P-AW-DMCS, CHROMOSORB 101, and CHROMOSORB 102, all available from Alltech Associates, Deerfield Ill.; ZSM-5 available from Amoco Chemical and Ca-montmorillonite, (Na,Ca)(Al,Mg)₆(Si₄O₁₀)₃(OH)₆.nH2O available under the tradename CARBOSORB SM 1502 from GSA Resources of Cortaro, Ariz. AVITENE is a commercially available blood coagulation accelerator material which consists of collagen. AVITENE was used for comparative purposes.

Additional blood coagulation accelerator materials were prepared in the laboratory. Materials synthesized in the laboratory include silica gel (SiO₂); alumina gel (Al₂O₃); (Na) zeolite 4A ((Na)₁₂[(AlO₂)₁₂(SiO₂)₁₂].27H₂O Y ((Na)₅₆(Al₅₆Si₁₃₆O₃₈₄).250H₂O)); (Ca) zeolite Y ((Ca, Na)₅₆(Al₅₆Si₁₃₆O₃₈₄).250H₂O)); (K) OMS-2 ((K)Mn₈O₁₆.nH₂O) (Ca) OMS-2 ((Ca, K)Mn₈O₁₆.nH2O); LDH, MgₓAl_{y}(OH)_{z}Clᵤ.nH₂O; chabazite (K₁₁(Al₁₁Si₂₅O₇₂).40H₂O); (Ca) OL-1 ((Ca,K,Na)Mn₁₄O₂₇.21H₂O); ZSM-5 (Na₇(Al₇Si₈₉O₁₉₂).nH₂O); (Ca) ZSM-5 ((Ca,Na)₇(Al₇Si₈₉O₁₉₂).nH₂O) zeolite RHO, (Na,Cs)₁₂[Al₁₂Si₃₆O₉₆].44H2O; Ca-mordenite, (Na-Ca)₅[Al₅Si₄₃O₉₆].nH₂O; silica-alumina, SiO₂-Al₂O₃; and silica-calcia SiO₂-CaO.

These materials were prepared as described below. Physical and chemical confirmations of the prepared materials were done using X-Ray Diffraction (XRD), Scanning Electron Microscopy (SEM) and Energy Dispersive X-Ray (EDX).

(Ca) OMS-2

500 milliliters (mL) of 1 molar calcium ion solution was prepared by dissolving calcium acetate or calcium chloride in the requisite quantity of water. 10 grams (gm) of (K) OMS-2 was added to the calcium ion solution and the mixture was stirred for twenty-four hours. After stirring, the mixture was filtered and the solids were washed using distilled water. The washed solids were dried in an oven at 100° C. for 24 hours.

(Ca) Zeolite Y

(Ca) zeolite Y was prepared by substituting 10 gm of (Na) zeolite Y for OMS-2 in the (Ca) OMS-2 procedure.

Silica Gel

Solution A (100 mL of water glass (SiO2/Na₂O=3.22)+200 mL of deionized distilled water (DDW)) and solution B (49 ml of 4.0 N HCl+60 mL of H₂O) were both cooled to about 5° C. Solution A was added to solution B with vigorous agitation. The resultant solution was poured into a flat tray to gel. After about 30 minutes the resulting stiff gel was cut into cubes. The cubes were transferred to a Buchner funnel and treated immediately with 1 N HCl for two hours. The HCl treatment procedure was twice more repeated. The gel was then washed free of chloride ions and subsequently dried for eight hours at 150° C. in an electric oven.

Alumina Gel

Solution A was prepared by dissolving 57 gram of AlCl₃.6H₂O in 1000 mL DDW. Solution B was prepared by diluting 84 mL of concentrated ammonium hydroxide to 145 mL DDW. Solution B was added to solution A with stirring. The precipitate was settled, filtered through a Buchner funnel and washed five times with very dilute ammonia solution (1 mL of concentrated ammonia+1000 mL DDW). Then the precipitate was dried for eight hours at 120° C. in an electric oven.

Zeolite 4A

Zeolite 4A was prepared according to the procedure described in Microiorous and Mesoporous Materials, 22:551-666, Robson, H. (editor), Elsevier, Amsterdam (1998); the disclosure of which is incorporated by reference herein.

(Na) Zeolite Y

Zeolite Y was prepared according to the procedure described in Microporous and Mesororous Materials, 22:551-666 (1998).

(K) OMS-2

11 grams (gm) of MnAc₂.6H2O in 40 mL DDW was dissolved in a buffer solution consisting of 5 mL of acetic acid and 5 gram of KAc in 40 mL of DDW. A solution of 6.5 gram of KMnO₄ in 150 mL was then added slowly with a burette. The resultant gel was refluxed with stirring for 24 hours. The product was filtered through a Buchner funnel, washed 3-5 times using 500 mL aliquots of DDW and dried for 24 hours at 110° C. in an electric oven.

Layered Double Hydroxide (LDH)

Layered double hydroxide material was prepared according to the procedure described in Miyata, S. Clays and Clay Materials, 23:369-375 (1975); the disclosure of which is incorporated by reference herein.

Chabazite

Chabazite was prepared according to the procedure described in Microporous and Mesoporous Materials, 22:551-666 (1998).

19 gm of MnCl2.4H2O and 6 gm of MgCl2.6H2O in 120 mL DDW were dissolved in a solution consisting of 30 gm NaOH and 150 mL DDW. A solution of 3.8 gm KMnO₄ in 140 mL DDW was added slowly with a burette. The product was kept for overnight at 60° C. in an electric oven. The precipitate was filtered through a Buchner funnel, washed 3-5 times using 500 mL DDW and dried for 24 hours at 110° C. in an electric oven.

(Ca) OL-1

Calcium type OL-1 was prepared by substituting 10 gm of OL-1 for OMS-2 in the (Ca) OMS-2 procedure.

(Ca) ZSM-5

Calcium type ZSM-5 was prepared by substituting ZSM-5 for OMS-2 in the (Ca) OMS-2 procedure.

Zeolite RHO

4 M Na₂AlO₂OH was prepared as follows: 294.3 gm of CATAPAL SB (available from Condea of Louisiana) and 320 gm of NaOH pellets were added to 800 mL H₂O and placed in oven at 100° C. for five days. After heating, the solution was cooled and diluted to 1 L. Solution A comprising 200 mL of 4 M Na₂AlO₂OH, 32 gm of NaOH pellets and 56 mL of 50% CsOH solution was prepared. 720 mL of LUDOX LS-30 (available from Dupont) was added to solution A. The mixture was shaken until it was homogeneous, and allowed to stand at room temperature for five days. The resultant solution was heated in an electric oven at 90° C. and shaken daily for 1-3 weeks.

(Ca) Mordenite

Mordrdenite was prepared according to the procedure described in Microporous and MesoDorous Materials, 22:551-666 (1998). Calcium type mordenite was prepared by substituting 10 gm of mordenite for OMS-2 in the (Ca) OMS-2 procedure.

Silica-Alumina

Solution A was prepared by dissolving 233 mL of water glass in 450 mL of DDW. Solution B was prepared by dissolving 34 mL 4 N HCl and 23 gm of Al₂(SO₄)₃.18H₂O in 200 mL DDW. Both solution A and solution B were cooled to about 5° C. Solution A was added to solution B rapidly with strong agitation. The resultant mixture was poured into a flat tray to gel and cut into cubes after one hour. The gel cubes were aged for 48 hours and transferred to a Buchner funnel. A 2% solution of Al₂(SO₄)₃.18H₂O was used to do the base exchange three times for 2-hour periods, then once overnight. The product was washed using DDW until free of sulfate ions and dried for eight hours at 170° C. in an electric oven.

Silica-Calcia

Silica-calcia was prepared according to the procedure described in Banal, N. P., J. Am. Ceram. Soc. 71(8):666-672 (1998); the disclosure of which is incorporated by reference herein.

It should be understood that the following examples are included for purposes of illustration so that the invention may be more readily understood and are in no way intended to limit the scope of the invention unless otherwise specifically indicated.

### EXAMPLE 1

The blood coagulation testing was performed using the following procedure. Silicon oxide treated sterile 7 ml vacutainer tubes were pre-weighed. A predetermined amount of each blood coagulation accelerator material was transferred into the pre-weighed tube. The pre-weighed tube, with material inside, was weighed and dried in an oven at 100° C. for at least 24 hours. Each tube was sealed with a septum in an inert gas atmosphere. The prepared tubes were allowed to cool, and weighed before and after use in the blood coagulation test.

20 mL syringes with needles were used to collect 10 mL of fresh blood from the jugular vein of a subject animal. 1 mL of the fresh blood was quickly dispensed to each pre-weighed tube containing a predetermined amount of blood coagulation accelerator material to give the ratio of material to blood indicated in the TABLES. The tubes were shaken to mix the blood and material together. Testing was done in batches of 6 test tubes at one time. Timing was started when the tube was initially filled with blood. After filling and mixing, each tube was monitored visually until a blood clot was seen. The coagulation time was recorded when the blood formed a solid clot with no flowing movement of the mixture observable within the tube.

The time required for blood clotting in a glass tube is a measure of the overall activity of the intrinsic system in blood coagulation. Periodic inspection of the clot permits evaluation of its physical properties (appearance, size and mechanical strength), its stability and the rate and extent of its retraction. See Hematology, William J. Williams, editor, 1661 McGraw-Hill (3rd edition, 1983), the entire disclosure of which is incorporated by reference herein.

Temperature changes of the tubes during testing were detected by a thermocouple attached to the external wall of the tube. Initial and maximum temperatures were recorded. The reaction between the blood sample and phosphorous pentoxide was severe enough to raise the blood sample to the boiling point. With the above exception, reactions between blood coagulation accelerator materials and the blood samples were within the range of −2° C. to 8° C. The temperature changes for some materials are summarized in TABLE 1.

Blank tubes that did not contain a blood coagulation accelerator material were used to establish a base line coagulation time for each sampling date and blood donor. This was done to minimize the influence of temperature, atmospheric and other environmental variables. Since the coagulation time of the blank tubes varied, a relative coagulation time was used to compare the effect of each blood coagulation accelerator material on blood clotting times. Relative coagulation time was calculated using the following equation.

Relative Coagulation Time=((Coagulation time of blood exposed to blood coagulation accelerator material)/(Coagulation time of blood in blank tube))×100.

Relative coagulation time is more precise than absolute coagulation time because environmental errors are lessened through the use of blank tests. The results of the individual blood coagulation tests are shown in TABLES 2 through 14. TABLE 16 is a compilation of Relative Coagulation Times for all materials. As can be seen from TABLE 16, many of the materials provide surprising and unexpected decreases in blood coagulation time. Given the present invention, other clays, zeolite materials, oxides and combinations thereof would also be expected to show similar advantageous effects in animal and human systems and their use is fully comprehended by the invention.

### EXAMPLE 2

Blood coagulation testing was also performed on mixtures of blood coagulation accelerator materials. The mixtures were prepared by mixing sodium aluminum oxide with another blood coagulation accelerator material that had proven individually to be effective in initiating and accelerating blood clotting. Each combination material contained approximately 50% sodium aluminum oxide and 50% blood coagulation accelerator material by weight. The mixtures of materials were prepared and tested using the above-described procedure for single blood coagulation accelerator materials. The results of the blood coagulation testing for mixed materials are shown in TABLE 15. As can be seen from TABLE 15, many of the mixed materials provide surprising and unexpected decreases in blood coagulation time when compared to the times for materials used alone.

### EXAMPLE 3

Certain calcium containing materials appear to be beneficial in promoting blood clotting. As shown in TABLE 16, powdered calcium oxide was found to speed blood clotting significantly. While not wishing to be held to a particular theory, the inventors believe that calcium ions can be essential for interaction with calcium ion dependent enzymes and blood clotting factors during homeostasis. The inventors also believe that calcium ions are important for platelet activation, activation of phospholipases, activation of calcium dependent proteases and other functions.

### EXAMPLE 4

Zeolite materials containing calcium cations were also tested for blood coagulation times. As can be seen from TABLE 12, the calcium exchanged versions of zeolite Y and OMS-2 were surprisingly more effective than the non-calcium exchanged version in accelerating blood coagulation. The capacity of zeolite materials to exchange ions is dependent on the size of the pores or channels therein, size of the ions, temperature and other factors. Materials such as (Na) zeolite Y and (Na) zeolite A can exchange the sodium ion inside their channels with calcium in the blood, removing calcium ions from the blood and retarding the coagulation process. With calcium exchanged zeolite materials there is no possibility of such removal of calcium ions from the blood, therefore blood coagulation is not retarded by the ion exchange. Not all calcium exchanged zeolite materials exhibit this beneficial behavior. As can be seen from TABLES 13 and 14, (Ca) ZSM-5, while advantageous in accelerating coagulation time, was somewhat less effective than non-calcium exchanged ZSM-5.

TABLE 1

| material | Temperature change (° C.) |
|---|---|
| P₂O₅ | Severe |
| Silica gel | 7.4 |
| Zeolite 4A | 7.7 |
| Zeolite Y | 6.0 |
| Zeolite 5A | 5.0 |
| Alumina gel | 4.6 |
| OMS-2 | 4.0 |
| (Ca) Zeolite Y | 3.5 |
| LDH | 2.9 |
| (Ca) OMS-2 | 1 |
| AVITENE | −2 |

TABLE 2
Test 1, Day 1, Horse Blood From Horse 2

| | | | Average | Relative | Test | Average test |
|---|---|---|---|---|---|---|
| | | Coagulation | Coagulation | Coagulation | material/blood | material/blood |
| Material | Sample # | Time | Time | Time % | ratio gm/ml | gm/ml |
| Blank | 1 | 00:25:38 | 00:30:15 | 100 | | |
| | 2 | 00:34:52 | | | | |
| OMS-2(Ca) + NaAlO₂ | 1 | 00:10:30¹ | 00:00:35 | 1.9 | 0.2941 | 0.3320 |
| | 2 | 00:00:35 | | | 0.3698 | |
| MgO + NaAlO₂ | 1 | 00:01:02 | 00:01:01 | 3.4 | 0.3967 | 0.3822 |
| | 2 | 00:01:00 | | | 0.3677 | |
| Zeolite Y(Ca) + NaAlO₂ | 1 | 00:01:41 | 00:01:09 | 3.8 | 0.3535 | 0.3660 |
| | 2 | 00:00:37 | | | 0.3785 | |
| Cerium Oxide + NaAlO₂ | 1 | 00:00:46 | 00:01:15 | 4.1 | 0.4308 | 0.4023 |
| | 2 | 00:01:44 | | | 0.3739 | |
| CaO + NaAlO₂ | 1 | 00:01:35 | 00:01:18 | 4.3 | 0.3075 | 0.3129 |
| | 2 | 00:01:01 | | | 0.3182 | |
| Silica Gel + NaAlO₂ | 1 | 00:01:41 | 00:01:28 | 4.8 | 0.3094 | 0.3324 |
| | 2 | 00:01:15 | | | 0.3553 | |

¹This result was considered abnormal and not used statistically.

TABLE 3
Test 2, Day 1, Cow Blood From Cow 1

| | | | Average | Relative | Test | Average test |
|---|---|---|---|---|---|---|
| | | Coagulation | Coagulation | Coagulation | material/blood | material/blood |
| Material | Sample # | Time | Time | Time % | ratio gm/ml | gm/ml |
| Blank | 1 | 00:13:24 | 00:13:23 | 100 | | |
| | 2 | 00:13:21 | | | | |
| NaAlO₂ | 1 | 00:00:27 | 00:00:25 | 3.1 | 0.2544 | 0.3039 |
| | 2 | 00:00:22 | | | 0.3534 | |
| OMS-2 | 1 | 00:00:33 | 00:00:32 | 4.0 | 0.4439 | 0.4031 |
| (Ca) | 2 | 00:00:30 | | | 0.3622 | |
| MgO | 1 | 00:00:39 | 00:00:38 | 4.7 | 0.3387 | 0.3270 |
| | 2 | 00:00:36 | | | 0.3152 | |
| CaO | 1 | 00:00:39 | 00:00:38 | 4.7 | 0.3777 | 0.3702 |
| | 2 | 00:00:36 | | | 0.3627 | |
| Silica Gel | 1 | 00:00:49 | 00:00:47 | 5.9 | 0.3135 | 0.3034 |
| | 2 | 00:00:44 | | | 0.2933 | |
| Cerium Oxide | 1 | 00:03:14 | 00:03:13 | 24 | 0.4661 | 0.4093 |
| | 2 | 00:03:11 | | | 0.3524 | |
| Zeolite 5A | 1 | 00:04:09 | 00:04:09 | 31 | 0.3391 | 0.3264 |
| | 2 | 00:04:08 | | | 0.3137 | |
| Zeolite Y | 1 | 00:07:49 | 00:07:33 | 56 | 0.3849 | 0.3864 |
| (Ca) | 2 | 00:07:16 | | | 0.3878 | |
| MgO + NaAlO₂ | 1 | 00:00:16 | 00:00:14 | 1.7 | 0.4663 | 0.4127 |
| | 2 | 00:00:11 | | | 0.3591 | |
| Silica Gel + NaAlO₂ | 1 | 00:00:30 | 00:00:27 | 3.4 | 0.3163 | 0.3450 |
| | 2 | 00:00:24 | | | 0.3744 | |
| Zeolite Y(Ca) + NaAlO₂ | 1 | 00:00:31 | 00:00:28 | 3.5 | 0.3998 | 0.3747 |
| | 2 | 00:00:25 | | | 0.3496 | |
| CaO + NaAlO₂ | 1 | 00:00:35 | 00:00:34 | 4.2 | 0.3381 | 0.3758 |
| | 2 | 00:00:32 | | | 0.4136 | |
| OMS-2(Ca) + NaAlO₂ | 1 | 00:00:41 | 00:00:40 | 4.9 | 0.2926 | 0.3552 |
| | 2 | 00:00:38 | | | 0.4178 | |
| Cerium Oxide + NaAlO₂ | 1 | 00:04:43 | 00:04:56 | 37 | 0.3447 | 0.3310 |
| | 2 | 00:05:09 | | | 0.3173 | |

TABLE 4
Test 3, Day 1, Dog blood from dog 1

| | | | Average | Relative | Test | Average test |
|---|---|---|---|---|---|---|
| | | Coagulation | Coagulation | Coagulation | material/blood | material/blood |
| Material | Sample # | Time | Time | Time % | ratio gm/ml | gm/ml |
| Blank | 1 | 00:07:49 | 00:08:02 | 100 | | |
| | 2 | 00:08:14 | | | | |
| NaAlO₂ | 1 | 00:00:34 | 00:00:33 | 6.9 | 0.2466 | 0.3425 |
| | 2 | 00:00:31 | | | 0.4384 | |
| OMS-2 | 1 | 00:00:35 | 00:00:34 | 7.0 | 0.3422 | 0.3619 |
| (Ca) | 2 | 00:00:32 | | | 0.3816 | |
| Silica Gel | 1 | 00:00:41 | 00:00:39 | 8.1 | 0.3839 | 0.3681 |
| | 2 | 00:00:37 | | | 0.3941 | |
| Cerium Oxide | 1 | 00:02:13 | 00:02:10 | 27 | 0.3780 | 0.3706 |
| | 2 | 00:02:07 | | | 0.3633 | |

TABLE 5
Test 4, Day 2, Dog Blood From Dog 1

| | | | Average | Relative | | Average test |
|---|---|---|---|---|---|---|
| | | Coagulation | Coagulation | Coagulation | Average Ratio | material/blood |
| Material | Sample # | Time | Time | Time % | Material/Blood | gm/ml |
| Blank | 1 | 0:04:36 | 0:05:05 | 100 | | |
| | 2 | 0:05:34 | | | | |
| CeO + NaAlO₂ | 1 | 0:00:04 | 0:00:04 | 1.31 | 0.3382 | 0.3371 |
| | 2 | 0:00:04 | | | | 0.3394 |
| CaO + NaAlO₂ | 1 | 0:00:38 | 0:00:37 | 12.13 | 0.3873 | 0.4082 |
| | 2 | 0:00:36 | | | | 0.3664 |
| CaO | 1 | 0:00:59 | 0:00:43 | 14.10 | 0.3858 | 0.3829 |
| | 2 | 0:00:27 | | | | 0.3886 |
| NaAlO₂ | 1 | 0:00:43 | 0:01:00 | 19.67 | 0.3636 | 0.3725 |
| | 2 | 0:01:17 | | | | 0.3546 |
| OMS-2 (Ca) + NaAlO₂ | 1 | 0:01:21 | 0:01:02 | 20.16 | 0.4244 | 0.383 |
| | 2 | 0:01:19 | | | | 0.4605 |
| Zeolite Y | 1 | 0:01:21 | 0:01:20 | 26.23 | 0.3847 | 0.3951 |
| (Ca) + | 2 | 0:01:19 | | | | 0.3743 |
| NaAlO₂ | | | | | | |
| Silica | 1 | 0:01:30 | 0:01:29 | 29.02 | 0.3332 | 0.3636 |
| Gel + NaAlO₂ | 2 | 0:01:27 | | | | 0.3028 |
| MgO + | 1 | 0:01:42 | 0:01:39 | 32.46 | 0:3215 | 0.2964 |
| NaAlO₂ | 2 | 0:01:36 | | | | 0.3466 |
| Zeolite Y | 1 | 0:01:53 | 0:01:51 | 36.23 | 0.3235 | 0.3190 |
| (Ca) | 2 | 0:01:48 | | | | 0.3279 |
| Zeolite 5A | 1 | 0:02:03 | 0:02:01 | 39.67 | 0:3301 | 0.3303 |
| | 2 | 0:01:59 | | | | 0.3299 |
| MgO | 1 | 0:01:59 | 0:05:34 | 109.67 | 0.3465 | 0.3488 |
| | 2 | 0:09:10 | | | | 0.3442 |

TABLE 6
Test 5, Day 3, Horse Blood From Horse 1

| | | | Average | Relative | | Average test |
|---|---|---|---|---|---|---|
| | | Coagulation | Coagulation | Coagulation | Average Ratio | material/blood |
| Material | Sample # | Time | Time | Time % | Material/Blood | gm/ml |
| Blank | 1 | 0:13:43 | 0:14:47 | 100 | | |
| | 2 | 0:14:16 | | | | |
| | 2 | 0:16:23 | | | | |
| NaAlO₂ | 1 | 0:01:06 | 0:00:53 | 6.01 | | 3.3882 |
| | 2 | 0:00:39 | | | 0.3853 | 0.3864 |
| | 3 | 0:00:55 | | | | 0.3813 |
| CaO | 1 | 0:01:45 | 0:01:21 | 9.09 | | 0.3909 |
| | 2 | 0:01:50 | | | 0.3880 | 0.3609 |
| | 3 | 0:00:27 | | | | 0.4121 |
| Cerium | 1 | 0:01:41 | 0:01:16 | 8.56 | 0.3756 | 0.3251 |
| Oxide/NaAlO₂ | 2 | 0:00:34 | | | | 0.3792 |
| | 3 | 0:01:33 | | | | 0.4226 |

TABLE 7
Test 6, Day 3, Horse Blood From Horse 2

| | | | Average | Relative | | Average test |
|---|---|---|---|---|---|---|
| | | Coagulation | Coagulation | Coagulation | Average Ratio | material/blood |
| Material | Sample # | Time | Time | Time % | Material/Blood | gm/ml |
| Blank | 1 | 0:12:56 | 0:15:51 | 100 | | |
| | 2 | 0:16:02 | | | | |
| | 2 | 0:18:36 | | | | |
| NaAlO₂ | 1 | 0:00:30 | 0:00:42 | 4.38 | 0.4160 | 0.4059 |
| | 2 | 0:00:43 | | | | 0.3989 |
| | 3 | 0:00:52 | | | | 0.4433 |
| CaO | 1 | 0:04:31 | 0:03:42 | 23.34 | 0.4035 | 0.3994 |
| | 2 | 0:02:16 | | | | 0.4247 |
| | 3 | 0:04:19 | | | | 0.3864 |

TABLE 8
Test 7, Day 3, Horse Blood From Horse 3

| | | Relative | Average | Average Ratio | | Average test |
|---|---|---|---|---|---|---|
| | | Coagulation | Coagulation | Material/ | Coagulation | material/blood |
| Material | Sample # | Time (%) | Time | Blood | Time | gm/ml |
| Blank | 1 | 100 | 0:17:27 | | 0:17:24 | |
| | 2 | | | | 0:15:25 | |
| | 3 | | | | 0:19:33 | |
| NaAlO₂ | 1 | 3.60 | 0:00:38 | 0.4020 | 0:00:58 | 0.3865 |
| | 2 | | | | 0:00:30 | 0.3746 |
| | 3 | | | | 0:00:25 | 0.4447 |
| CaO | 1 | 22.53 | 0:03:56 | 0.3371 | 0:04:02 | 0.2996 |
| | 2 | | | | 0:04:11 | 0.3533 |
| | 3 | | | | 0:03:35 | 0.3585 |

TABLE 9
Test 8, Day 3, Dog Blood From Dog 1

| | | Relative | Average | Average Ratio | | Average test |
|---|---|---|---|---|---|---|
| | | Coagulation | Coagulation | Material/ | Coagulation | material/blood |
| Material | Sample # | Time (%) | Time | Blood | Time | gm/ml |
| Blank | 1 | 100 | 0:05:53 | | 0:05:54 | |
| | 2 | | | | 0:05:36 | |
| | 3 | | | | 0:06:10 | |
| NaAlO₂ | 1 | 9.53 | 0:00:34 | 0.3703 | 0:00:37 | 0.3413 |
| | 2 | | | | 0:00:34 | 0.3849 |
| | 3 | | | | 0:00:30 | 0.3847 |
| CaO | 1 | 6.13 | 0:00:22 | 0.3966 | 0:00:24 | 0.3654 |
| | 2 | | | | 0:00:21 | 0.4442 |
| | 3 | | | | 0:00:20 | 0.3801 |

TABLE 10
Test 9, Day 3, Dog Blood From Dog 2

| | | Relative | Average | Average Ratio | | Average test |
|---|---|---|---|---|---|---|
| | | Coagulation | Coagulation | Material/ | Coagulation | material/blood |
| Material | Sample # | Time (%) | Time | Blood | Time | gm/ml |
| Blank | 1 | 100 | 0:05:54 | | 0:05:37 | |
| | 2 | | | | 0:06:04 | |
| | 3 | | | | 0:06:01 | |
| NaAlO₂ | 1 | 10.73 | 0:00:38 | 0.3906 | 0:00:42 | 0.4039 |
| | 2 | | | | 0:00:38 | 0.3961 |
| | 3 | | | | 0:00:34 | 0.3718 |
| CaO | 1 | 9.98 | 0:00:35 | 0.4646 | 0:00:17 | 0.4672 |
| | 2 | | | | 0:00:51 | 0.4154 |
| | 3 | | | | 0:00:38 | 0.5110 |

TABLE 11
Test 10, Day 3, Dog blood from dog 3

| | | Relative | Average | Average Ratio | | Average test |
|---|---|---|---|---|---|---|
| | | Coagulation | Coagulation | Material/ | Coagulation | material/blood |
| Material | Sample # | Time (%) | Time | Blood | Time | gm/ml |
| Blank | 1 | 100 | 0:07:42 | | 0:04:43 | |
| | 2 | | | | 0:09:10 | |
| | 3 | | | | 0:09:13 | |
| NaAlO₂ | 1 | 8.59 | 0:00:40 | 0.3599 | 0:00:38 | 0.3247 |
| | 2 | | | | 0:00:42 | 0.3862 |
| | 3 | | | | 0:00:39 | 0.3689 |
| CaO | 1 | 8.87 | 0:00:41 | 0.3825 | 0:00:48 | 0.3734 |
| | 2 | | | | 0:00:51 | 0.3782 |
| | 3 | | | | 0:00:24 | 0.3959 |

TABLE 12
Test 11, Day 4, Horse Blood From Horse 2

| | | | Average | Relative | | Average test |
|---|---|---|---|---|---|---|
| | | Coagulation | Coagulation | Coagulation | Average Ratio | material/blood |
| Material | Sample # | Time | Time | Time % | Material/Blood | gm/ml |
| Blank | 1 | 0:41:22 | 0:41:19 | 100 | | |
| | 2 | 0:41:17 | | | | |
| ZSM-5 | 1 | 0:01:24 | 0:01:22 | 3.3 | 0.3849 | 0.3828 |
| | 2 | 0:01:23 | | | | 0.3997 |
| | 3 | 0:01:20 | | | | 0.3720 |
| (Ca) ZSM-5 | 1 | 0:03:33 | 0:04:16 | 10.3 | 0.3767 | 0.3744 |
| | 2 | 0:04:01 | | | | 0.3294 |
| | 3 | 0:05:13 | | | | 0.4263 |

TABLE 13
Test 12, Day 4, Dog Blood From dog 1

| | | | Average | Relative | | Average test |
|---|---|---|---|---|---|---|
| | | Coagulation | Coagulation | Coagulation | Average Ratio | material/blood |
| Material | Sample # | Time | Time | Time % | Material/Blood | gm/ml |
| Blank | 1 | 0:15:38 | 0:15:35 | 100 | | |
| | 2 | 0:15:31 | | | | |
| ZSM-5 | 1 | 0:01:30 | 0:01:25 | 9.1 | 0.3924 | 0.3679 |
| | 2 | 0:01:25 | | | | 0.3985 |
| | 3 | 0:01:20 | | | | 0.4108 |
| (Ca) ZSM-5 | 1 | 0:01:41 | 0:02:00 | 12.8 | 0.3809 | 0.4333 |
| | 2 | 0:02:03 | | | | 0.4182 |
| | 3 | 0:02:15 | | | | 0.2913 |

| | COMPARATIVE TABLE 14 | | | | | |
|---|---|---|---|---|---|---|
| | Relative Coagulation Time (%) at specified test material/blood ratio (gm/ml) | | | | | |
| | Horse Blood | | | | Cow Blood | Dog Blood |
| Mixed Material | 0.4 g/ml | 0.2 g/ml | 0.1 g/ml | 0.05 g/ml | 0.4 g/ml | 0.4 g/ml |
| AVITENE | | | | 8.2 | | |
| SiO₂/NaAIO₂ | 4.8 | | | | 3.4 | 29.02 |
| OMS-2(Ca)/ | 1.9 | | | | 4.9 | 20.16 |
| NaAIO₂ | | | | | | |
| MgO/NaAIO₂ | 3.4 | | | | 1.7 | 32.5 |
| CaO/NaAIO₂ | 4.3 | | | | 4.2 | 12.1 |
| CeO/NaAIO₂ | 4.1 | | | | 37.0 | 1.31 |
| Ca-ZeoliteY/ | 3.8 | | | | 3.5 | 26.2 |
| Y/NaAIO₂ | | | | | | |

| | COMPARATIVE TABLE 15 | | | | | |
|---|---|---|---|---|---|---|
| | Relative Coagulation Time (%) | | | | | |
| | Horse Blood | | | | Cow Blood | Dog Blood |
| Material | 0.4 g/ml | 0.2 g/ml | 0.1 g/ml | 0.05 g/ml | 0.4 g/ml | 0.4 g/ml |
| AVITENE | | | | 8.2 | | |
| OMS-2 (K) | | 59.4 | 117.8 | 137.0 | | |
| OMS-2 (Ca) | 3.3, 6.6 | 18.6 | | 106.8 | 8.2, 4.0 | 7.0 |
| Zeolite Y (Na) | | >100 | >100 | >100 | | |
| Zeolite Y (Ca) | 9.7, 30.7 | 16.5 | 36.9 | | 35.4, 56 | 36.2 |
| Ca-OL-1 | 11.2 | >100 | | | 49.0 | |
| Zeolite ZSM-5 | 3.3 | | | | | 9.1 |
| Zeolite ZSM-5 | 10.3 | | | | | 12.8 |
| (Ca) | | | | | | |
| Montmorillonite | | | | | | |
| Ca- | 21.5 | 23.7 | | | 20 | |
| Montmorillonite | | | | | | |
| Mordenite | | | | | | |

| | COMPARATIVE TABLE 15 | | | | | |
|---|---|---|---|---|---|---|
| | Relative Coagulation Time (%) | | | | | |
| | Horse Blood | | | | Cow Blood | Dog Blood |
| Material | 0.4 g/ml | 0.2 g/ml | 0.1 g/ml | 0.05 g/ml | 0.4 g/ml | 0.4 g/ml |
| Ca-Mordenite | 41.9 | 26.7 | | | | |
| Zeolite 4A (Na) | | >100 | 273 | 372 | | |
| Commercial | | | | | | |
| Sample | | | | | | |
| Zeolite 4A (Na) | | 363.8 | 420.5 | 338.5 | | |
| Lab Sample | | | | | | |

| | COMPARATIVE TABLE 16 | | | | | |
|---|---|---|---|---|---|---|
| | Relative Coagulation Time (%) | | | | | |
| | Horse Blood | | | | Cow Blood | Dog Blood |
| Material | 0.4 g/ml | 0.2 g/ml | 0.1 g/ml | 0.05 g/ml | 0.4 g/ml | 0.4 g/ml |
| AVITENE | | | | 8.2 | | |
| Silica Gel | 2.5, 1.8, 1.6 | 6.95, 1.97 | 10.34 | 9.27 | 19.6, 5.9 | 8.1 |
| (SiO₂)¹ | | | | | | |
| CHROMOSORB | 2.6 | | | | 47.1 | |
| PAW-DMCS | | | | | | |
| Silica Gel w/o | 2.8 | | | | | |
| Dehydration ¹ | | | | | | |
| Chromatographic | 4.7 | | | | | |
| Silica Gel ¹ | | | | | | |
| Acid Silica | 13.5 | | | | | |
| (SiO₂nH₂O | | | | | | |
| OMS-2 (Ca) | 3.3, 6.6 | 18.6 | | 106.8 | 8.2, 4.0 | 7.0 |
| NaAlO₂ | 3.8 | | | | 12.1, 3.1 | 6.9, 19.7 |
| MgO | 3.9 | >100 | | | 12.3, 4.7 | 109.7 |
| Cerium Oxide | 22.4 | | | | 18.3, 24 | 27 |
| CaO | 6.1 | 8.4 | | | 12.3, 4.7 | 14.1 |
| P₂O₅ | 6.2 | | | | 6.4 | |
| Ca-Zeolite Y | 9.7, 30.7 | 16.5 | 36.9 | | 35.4, 56 | 36.2 |
| Chabazite | 2.1, 46.2 | 76.8 | | | 89.5 | |
| Ca-OL-1 | 11.2 | >100 | | | 49.0 | |
| Zeolite ZSM-5 | 3.3 | 30.9 | | | | 9.1 |
| Ca-Montmorillonite | 21.5 | 23.7 | | | 20 | |
| Bismuth Oxide | 20.9 | | | | 46.3 | |
| Silica Alumina | 21.3 | 48.6 | | | | |
| Activated | 27.0 | 29.0 | | | 43.3 | |
| Carbon | | | | | | |
| Zeolite 5A | 41.3, 23.2 | 68.8 | 52.9 | 69.87 | 26.4, 31 | 39.7 |
| Zeolite RHO | 30.5 | 23.2 | | | | |
| Ca-Mordenite | 41.9 | 26.7 | | | | |
| Ferric Oxide | 31.9 | | | | | |
| Chromosorb 101 | 34.2 | | | | | |
| Yttrium Oxide | 37.6 | | | | | |
| Cobalt Oxide | 38.3 | | | | | |
| Calcium | 38.3 | | | | | |
| Phosphate | | | | | | |
| Chromosorb 102 | 41.2 | | | | | |
| Stannic Oxide | 68.13 | | | | | |
| Yunnan White | | | | 78.1 | | |
| Medicine | | | | | | |
| Nickel Oxide | 85.7 | | | | | |
| Copper Oxide | 91.5 | | | | | |
| Yb₂O₃ | 97.3 | | | | | |
| OMS-2 (K) | | 59.4 | 117.8 | 137.0 | | |
| Alumina Gel | | 82.3 | 161 | 131 | | |
| Zeolite Na-Y | | >100 | >100 | >100 | | |
| Zeolite 4A (Na)- | | >100 | 273 | 372 | | |
| Commercial | | | | | | |
| Sample | | | | | | |
| LDH | | 157.4 | | 165.6 | | |
| Coffee Powder | | | | 261.8 | | |
| Zeolite 4A (Na)-Lab | | 363.8 | 420.5 | 338.5 | | |
| Ca-ZSM-5 | 10.3 | | >100 | | | 12.8 |
| Silica-Calcia | >100 | >100 | | | | |
| Calcium | >100 | | | | | |
| Chloride | | | | | | |
| Barium Oxide | >100 | | | | | |
| Aluminum | >100 | | | | | |
| Oxide | | | | | | |
| Zinc Oxide | >100 | | | | | |
| Vanadium | >100 | | | | | |
| Pentoxide | | | | | | |
| Titanic Oxide | >100 | | | | | |
| Zirconium | >100 | | | | | |
| Oxide | | | | | | |
| Scandium | >100 | | | | | |
| Oxide | | | | | | |
| Europium | >100 | | | | | |
| Oxide | | | | | | |
| LiAlO₂ | >100 | | | | | |

### EXAMPLE 5

Blood coagulation accelerator materials were coated onto porous flexible substrates; non-porous flexible substrates; and rigid substrates. The coated substrates were prepared using the following procedures.

Coatings on Porous Flexible Substrates

A solution of (Na) Zeolite Y was prepared. A swatch of cotton fabric approximately two centimeters (cm) by two centimeters (cm) was submerged in the solution without agitation for 5 minutes. The soaked swatch was removed from the solution, placed in an autoclave set at 100° C. and heated for a first time period of 24 hours. After the first heating period, the coated swatch was washed in distilled water and dried in an oven set at 100° C. for a second time period of 24 hours.

Coatings on Non-Porous Flexible Substrates

A film of siloxane was prepared by applying room temperature vulcanizing silicone sealant (500 RTV HIGH HEAT RUTLAND SILICONE SEALER available from Rutland Products, Rutland Vt.) over a non-stick surface. The film was allowed to cure at room temperature and in air for 24 hours, after which the 20 cured film was removed from the surface, washed in water and dried with a paper towel.

A gel of Zeolite 4A was prepared as previously described. The prepared Zeolite 4A gel was applied to the cured siloxane film using a spatula. The coated siloxane film was placed into a fluorocarbon bottle and heated at 80° C. for 3 hours. The resulting coated siloxane film was washed with distilled water and dried at 100° C. for 4 hours.

Coatings on Rigid Substrates

In a first test, sodium aluminum oxide was mixed individually with other blood coagulation accelerator materials in water to create a paste. Each paste mixture was coated on a wooden substrate and dried at 100° C. for 24 hours.

In a second test, polyvinyl acetate (FLEXBOND 153 EMULSION, available from Air Products and Chemicals Incorporated of Allentown, Pa.) was applied onto a wooden substrate and allowed to stand for approximately 15 minutes. Blood coagulation accelerator materials were sprinkled liberally onto individual polyvinyl acetate coated wooden substrates at the end of the 15 minute period. Excess particles of the material were shaken off and the coated substrate was dried at 100° C. for 24 hours.

In both tests, the coating strengths were determined by weighing the dried substrate and blood coagulation accelerator material, applying adhesive tape over the coating, peeling the tape off, and recording the weight of the blood coagulation accelerator material and test substrate remaining after the tape had been peeled off. No weight change was observed for coatings of (Ca) ZMS-5, Zeolite 5A or silica gel, indicating no material was removed from the substrate.

In every case it was found that blood coagulation accelerator materials could be successfully coated onto a substrate.

### EXAMPLE 6

Small packages or sachets each containing a blood coagulation accelerator material were prepared. Each package was approximately 1.5 cm by 1.5 cm and comprised an outer cover or wrap of a nonwoven material enclosing about 0.1 gm of blood coagulation accelerator material. The package functioned as a support for the blood coagulation accelerator material. KIMWIPES EX-L wiper material available from Kimberly-Clark was found suitable for use as the nonwoven material. The packages with blood coagulation accelerator material enclosed within were dried at 100° C. for 24 hours. After drying, the packages were individually sealed in glass vials until use.

Anesthetized rats were used as test subjects. The three nails of each rear foot were simultaneously clipped at the juncture of the nail and the skin to induce bleeding. The nails of the left foot were left untreated while the nails of the right foot were treated with a blood coagulation accelerator material as described below. Just prior to nail clipping, a vial containing a dried sachet of blood coagulation accelerator material was opened. After the nails were clipped, the sachet was opened and the nails of the right foot were inserted into the sachet and immersed in the blood coagulation accelerator materials. The right foot was removed from the sachet at 30-second intervals and the clotting processes of the untreated (left foot) and treated (right foot) nails were observed. Blood-clotting time, e.g. cessation of bleeding, was noted, and a relative and average relative clotting time were calculated from the data.

Relative clot time is calculated from:

((treated clot time)/(untreated clot time))×100.

Mean relative clot time is calculated from:

(Sum of relative clot times for each material)/(number of tests).

The results of the testing are shown in TABLE 17. As shown by the results, the treatment with the blood coagulation accelerators substantially shortened the time required for bleeding to stop in each test.

TABLE 17

| | | | Treated | Relative | Mean |
|---|---|---|---|---|---|
| | | Untreated | clot | clot | Relative |
| | material | clot time | time | time | clot time |
| 1 | MgO | 930 | 120 | 12.9 | 38.0 |
| 2 | MgO | 900 | 210 | 23.3 | |
| 3 | MgO | 450 | 240 | 53.3 | |
| 4 | MgO | 960 | 360 | 37.5 | |
| 5 | MgO | 780 | 420 | 53.8 | |
| 6 | MgO | 960 | 450 | 46.9 | |
| 1 | (Ca) Zeolite Y | 870 | 330 | 37.9 | 36.4 |
| 2 | (Ca) Zeolite Y | 1290 | 270 | 20.9 | |
| 3 | (Ca) Zeolite Y | 630 | 300 | 47.6 | |
| 4 | (Ca) Zeolite Y | 1920 | 330 | 17.2 | |
| 5 | (Ca) Zeolite Y | 1290 | 360 | 27.9 | |
| 6 | (Ca) Zeolite Y | 450 | 300 | 66.7 | |
| 1 | Silica gel | 570 | 210 | 36.8 | 28.0 |
| 2 | Silica gel | 630 | 90 | 14.3 | |
| 3 | Silica gel | 930 | 150 | 16.1 | |
| 4 | Silica gel | 1200 | 360 | 30.0 | |
| 5 | Silica gel | 1260 | 330 | 26.2 | |
| 6 | Silica gel | 540 | 240 | 44.4 | |
| 1 | (Ca) Oms-2 | 630 | 60 | 9.5 | 26.9 |
| 2 | (Ca) Oms-2 | 720 | 120 | 16.7 | |
| 3 | (Ca) Oms-2 | 600 | 150 | 25.0 | |
| 4 | (Ca) Oms-2 | 480 | 330 | 68.8 | |
| 5 | (Ca) Oms-2 | 1200 | 240 | 20.0 | |
| 6 | (Ca) Oms-2 | 840 | 180 | 21.4 | |

While preferred embodiments of the foregoing invention have been set for purposes of illustration, the foregoing description should not be deemed imitation of the invention herein. Accordingly, various modifications, adaptations and alternatives may occur to one skilled in the art without departing the spirit and scope of the present invention.

What is claimed is:

1. A method of accelerating a coagulation time of blood comprising:
providing means for accelerating blood coagulation; and
applying said means for accelerating blood coagulation to said blood.

2. The method of claim 1, wherein said means for accelerating blood coagulation is selected from the group consisting of calcium cations, an inorganic oxide, an inorganic oxide in combination with calcium cations, a clay, a clay in combination with calcium cations, a zeolite in combination with calcium cations and a zeolite in combination with an inorganic oxide

3. The method of claim 1, wherein said blood is flowing from a wound in a circulatory system and said step of applying comprises applying said means for accelerating blood coagulation to said wound.

4. The method of claim 1, wherein said means for accelerating blood coagulation consists essentially of an inorganic oxide and calcium cations.

5. The method of claim 1, wherein said means for accelerating blood coagulation consists essentially of an inorganic oxide.

6. The method of claim 1, wherein said means for accelerating blood coagulation is selected from the group consisting of calcium cations, an inorganic oxide, an inorganic oxide in combination with calcium cations, a zeolite in combination with calcium cations and a zeolite in combination with an inorganic oxide and said inorganic oxide is selected from the group consisting of aluminum oxide, bismuth oxide, calcium oxide, cerium oxide, cobalt oxide, ferric oxide, magnesium oxide, sodium aluminum oxide, nickel oxide, phosphorous pentoxide, stannic oxide, yttrium oxide, ytterbium oxide, silica gel, acid silica and combinations thereof.

7. The method of claim 1, wherein said means for accelerating blood coagulation consists essentially of a combination of zeolites and inorganic oxides.

8. The method of claim 1, wherein said means for accelerating blood coagulation is disposed on a support.

9. The method of claim 1, wherein said means for accelerating blood coagulation is contained within a package.

10. The method of claim 1, wherein said blood after applying said means for accelerating blood coagulation has a relative coagulation time within the range of 1.6 to 97.3.

11. The method of claim 1, wherein said blood after applying said means for accelerating blood coagulation has a relative coagulation time within the range of 1.6 to 24.

12. A wound dressing comprising:
a support; and
a blood coagulation accelerator disposed on said support, said blood coagulation accelerator selected from the group consisting of calcium cations, an inorganic oxide, an inorganic oxide in combination with calcium cations and a zeolite in combination with an inorganic oxide.

13. The wound dressing of 12 wherein said zeolite comprises OL-1, OL-1(Ca), OMS-2(K), OMS-2(Ca), Zeolite Y, Zeolite Y(Ca), Zeolite ZSM-5, Zeolite 5A and Zeolite RHO.

14. The wound dressing of claim 12 wherein said inorganic oxide comprises bismuth oxide, calcium oxide, cerium oxide, cobalt oxide, ferric oxide, magnesium oxide, sodium aluminum oxide, phosphorous pentoxide, yttrium oxide, silica gel, acid silica or combinations thereof.

15. A material for reducing a coagulation time of blood, said material selected from the group consisting of calcium cations, an inorganic oxide, an inorganic oxide in combination with calcium cations, a zeolite in combination with calcium cations, a zeolite in combination with an inorganic oxide and mixtures thereof.

16. The material of claim 15, wherein a relative coagulation time within the range of about 1.6 to 98 is obtained when said material and said blood are mixed at a ratio of about 0.4 grams of said material for each milliliter of blood.

17. The material of claim 15, wherein a relative coagulation time within the range of about 2.0 to 30 is obtained when said material and said blood are mixed at a ratio of about 0.2 grams of material for each milliliter of blood.

18. The material of claim 15, wherein a relative coagulation time within the range of about 10 to 40 is obtained when said material and said blood are mixed at a ratio of about 0.1 grams of said material for each milliliter of blood.

19. The material of claim 15, which when mixed with said blood at a ratio of about 0.4 grams of said material for each milliliter of blood will reduce said blood coagulation time by a factor within the range of 0.5 to 0.02.
